(19) United States
(12) Patent Application Publication
(10) Pub. No.: US 2026/0001768 A1
(43) Pub. Date: Jan. 1, 2026

(54) SYNTHESIS OF TETRADECANOIC MODIFIED GRAPHENE AS A CORROSION INHIBITOR

(71) Applicants: SAUDI ARABIAN OIL COMPANY, Dhahran (SA); KING FAHD UNIVERSITY OF PETROLEUM & MINERALS, Dhahran (SA)

(72) Inventors: Bader G. Alharbi, Dammam (SA); Norah A. Aljeaban, Al Khobar (SA); Tawfik A. Saleh, Dhahran (SA); Wajdi M. Buhaezah, Dhahran (SA)

(73) Assignees: SAUDI ARABIAN OIL COMPANY, Dhahran (SA); KING FAHD UNIVERSITY OF PETROLEUM & MINERALS, Dhahran (SA)

(21) Appl. No.: 19/334,167

(22) Filed: Sep. 19, 2025

Related U.S. Application Data
(62) Division of application No. 18/173,445, filed on Feb. 23, 2023.

(51) Int. Cl. C01B 32/194; C01B 32/198; C01B 32/23; C07D 493/22; C09K 15/22

(52) U.S. Cl. CPC C01B 32/194; C01B 32/198; C01B 32/23; C07D 493/22; C09K 15/22

(57) ABSTRACT

Described is a method of synthesizing a corrosion inhibitor. An amine modified graphene oxide is synthesized from graphene oxide nanosheets using a bifunctional amine having a terminal amide group. Amide groups of the bifunctional amine are reacted with carboxyl groups of the graphene oxide. Aliphatic and amine modified graphene oxide is synthesized from the amine modified graphene oxide using an aliphatic acid. The aliphatic and amine modified graphene oxide includes aliphatic groups derived from a long chain aliphatic acid and amine groups derived from the bifunctional amine. Amine modified graphene oxide is synthesized by dispersing graphene oxide nanosheets in methanol, adding diethylenetriamine, and adding an amount of N,N′-Dicyclohexylcarbodiimide (DCC) catalyst. Tetradecanoic and amine modified graphene nanosheets are synthesized by adding amine modified graphene to tetradecanoic acid to form a solution, then adding an amount of cobalt-salen catalyst to the solution.

### BACKGROUND

Carbon steel corrosion has great economic and environmental implications due to its applications in many construction industries. It is sometimes difficult to prevent petroleum from flowing quickly into the wellbore because it is present in formations with exceptionally low permeability. Low permeability is typically solved by stimulating the well, which opens up new channels and enlarges existing ones. The most successful and widely used method for stimulating oil and gas wells to increase output is acidizing. Acidizing entails injecting a hot acid solution into the wellbore. Acidizing eliminates mud from new wells and develops channels in rocks to allow oil to flow into the wells. Additionally, acidizing restores the maximum productivity of aged wells by dissolving debris. However, the acidizing process exposes the well's carbon steel construction to a highly corrosive acid medium. To lessen the acid assault on steel, corrosion inhibitors are applied as chemical additives as an alternative to acidizing.

Some of the effective corrosion inhibitors that are commercially available include formulations of aromatic aldehydes, quaternary salts, acetylenic alcohols, N-containing heterocyclics, carbonyls, and amines. These inhibitors are costly, poisonous, unfriendly to the environment, and only work at extremely high concentrations. Therefore, it is necessary to identify new inhibitors that are inexpensive, efficient, and environmentally friendly.

Metals have been protected against corrosion by graphene derivatives in a variety of situations. Comparing graphene to more traditional protection strategies, such as the use of polymeric coatings and inert metals, graphene has the benefit of being less expensive and more environmentally benign. Increased mechanical strength, large surface area, excellent chemical resistance, and a high level of impermeability are all characteristics of graphene. However, due to the functional limitations of graphene, it is not soluble in aqueous solution. Thus, its modification is essential to improve its properties.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a method of synthesizing a corrosion inhibitor by synthesizing amine modified graphene oxide from graphene oxide using a bifunctional amine comprising a terminal amide group.

In another aspect, aliphatic and amine modified graphene oxide is synthesized from the amine modified graphene oxide using an aliphatic acid.

In another aspect, the aliphatic and amine modified graphene oxide includes aliphatic groups derived from the long chain aliphatic acid and amine groups derived from the bifunctional amine.

In another aspect, the aliphatic and amine modified graphene oxide includes long chain aliphatic and amine modified graphene oxide nanosheets.

In yet another aspect, the graphene oxide comprises graphene oxide nanosheets.

In another aspect, the graphene oxide is prepared from graphite.

In another aspect, amide groups of the bifunctional amine are reacted with carboxyl groups of the graphene oxide to synthesize the amine modified graphene oxide.

In yet another aspect, the bifunctional amine is straight chain.

In another aspect, the bifunctional amine includes diethylenetriamine.

In another aspect, acid groups of the aliphatic acid are reacted with ether groups of the amine modified graphene oxide to synthesize aliphatic and amine modified graphene oxide.

In yet another aspect, the aliphatic acid includes a C6-C20 acid.

In another aspect, the aliphatic acid includes tetradecanoic acid.

Other aspects and advantages of the claimed subject matter will be apparent from the following description and the appended claims.

### BRIEF DESCRIPTION OF DRAWINGS

FIG. 1 is an illustration of the synthesis of graphene nanosheets from graphite according to embodiments of the present disclosure.

FIG. 2 is an illustration of the synthesis of amine modified graphene from graphene oxide according to embodiments of the present disclosure.

FIG. 3 is the synthesis of tetradecanoic and amine modified graphene nanosheets according to embodiments of the present disclosure.

FIG. 4 is an illustration of the mechanism of the formation of tetradecanoic branches via epoxy groups on graphene according to embodiments of the present disclosure.

### DETAILED DESCRIPTION

In one aspect, embodiments disclosed herein relate to a method of synthesizing a corrosion inhibitor. The method includes synthesizing amine modified graphene oxide from graphene oxide using a bifunctional amine comprising a terminal amide group.

According to one or more embodiments of the present disclosure, graphene oxide nanosheets may be synthesized from graphite. Graphite oxide nanosheets may be prepared via chemical and thermal treatment and a modified Hummer's method. Graphite may be used as a precursor to prepare the graphene oxide nanosheets. Graphene oxide nanosheets may be prepared by a wet chemical procedure using waste graphite powder. In some embodiments, a mixture of graphite powder and potassium permanganate (KMnO₄) is slowly added to an ice-cold mixture of sulfuric acid (H₂SO₄) and phosphoric acid (H₃PO₄) while stirring. The mixture is heated under stirring conditions and then allowed to cool overnight before being poured into deionized ice water.

In some embodiments, hydrogen peroxide (H₂O₂) may be slowly added under stirring conditions until a yellowish product is formed. The resulting product is allowed to settle overnight before the supernatant is removed. The remaining product is then washed several times with water to eliminate any remaining acid. The residual material may be washed with 10% hydrochloric acid (HCl) and distilled water to remove unreacted metal ions. The final product is dissolved in deionized water, where the graphene oxide dissolves and unreacted graphite settles. The dissolved graphene oxide is decanted, centrifuged, and allowed to dry into graphene oxide nanosheets.

According to some embodiments of the present disclosure, amine modified graphene is synthesized from the graphene oxide nanosheets. The synthesis of diethylenetriamine modified graphene oxide, or amine modified graphene may be achieved by dispersing graphene oxide nanosheets in methanol under sonication. A predetermined amount of diethylenetriamine is added under stirring conditions, and N,N′-Dicyclohexylcarbodiimide (DCC) is added as a catalyst. Thereafter, the system is allowed to cool to with continuous stirring. After cooling, amine modified graphene is separated by a centrifuge.

In one or more embodiments, tetradecanoic and amine modified graphene nanosheets inhibitor is synthesized as follows. The obtained amine modified graphene is added into tetradecanoic acid under stirring. Then, predetermined amounts of cobalt-salen catalyst are added. The product is collected and dried to produce the tetradecanoic and amine modified graphene nanosheets.

### EXAMPLES

### Example 1—Synthesis of Graphene

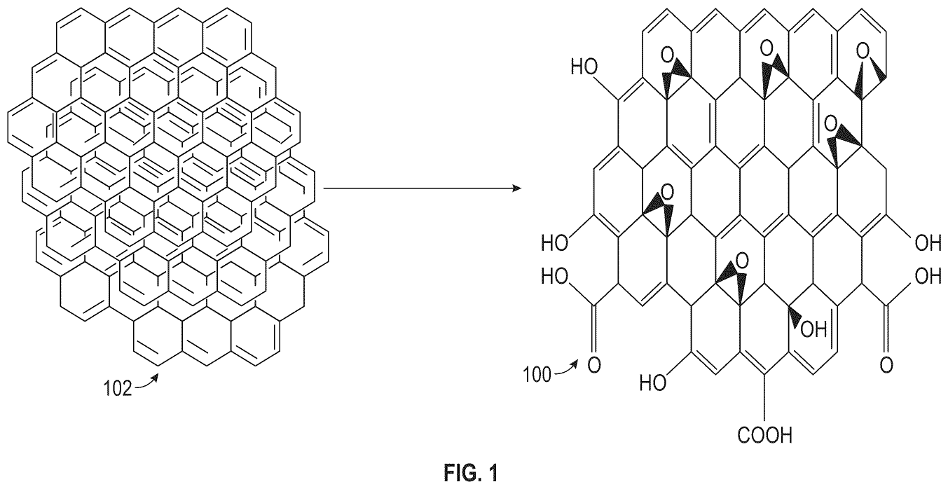

FIG. 1 depicts the synthesis of graphene oxide nanosheets 100 from graphite 102. Graphene oxide nanosheets 100 were prepared via chemical and thermal treatment and a modified Hummer's method. As shown, graphite 102 was used as a precursor to prepare the graphene oxide nanosheets 100. Graphene oxide nanosheets 100 were prepared by a wet chemical procedure using waste graphite powder. A mixture of approximately 8 to 100 grams (g), such as 10 g, of graphite powder and approximately 25 g to 230 g, such as 30 g, of potassium permanganate (KMnO₄) was slowly added to an ice-cold mixture of approximately 400 to 1000 milliliters (ml), such as 500 ml, of 96% sulfuric acid (H₂SO₄) and approximately 40 to 120 ml, such as 50 ml, of phosphoric acid (H₃PO₄) while stirring for a predetermined amount of time. The mixture was heated for approximately 24 hours at approximately 50 degrees Celsius (° C.) under stirring conditions. The heated mixture was then allowed to cool at ambient temperature overnight before being poured into approximately 800 mL of deionized ice water.

Approximately 100 ml of hydrogen peroxide (H₂O₂) (30%) was slowly added to the cooled solution under stirring conditions until a yellowish product is formed. The resulting product was allowed to settle overnight before the supernatant was removed. The remaining product was then washed several times with water to eliminate any remaining acid. The residual material was washed approximately three times with 10% hydrochloric acid (HCl) and distilled water to remove unreacted metal ions. The final product was dissolved in deionized water, where the graphene oxide dissolved, and unreacted graphite settled. The dissolved graphene oxide was decanted, centrifuged for approximately one hour at 1000 revolutions per minute (rpm), and allowed to dry.

### Example 2—Synthesis of Amine Modified Graphene

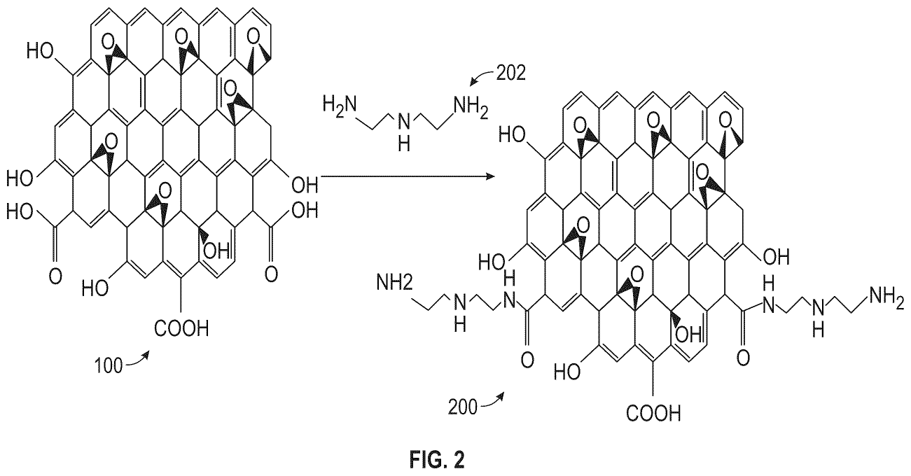

FIG. 2 illustrates the synthesis of amine modified graphene oxide 200 from graphene oxide nanosheets 100. The synthesis of diethylenetriamine modified graphene oxide, or amine modified graphene oxide 200, was achieved by the following procedure. One gram of graphene oxide nanosheets 100 was dispersed in about 100 to 300 ml of methanol under sonication for approximately two hours (h). Then, diethylenetriamine 202 was added in an amount of 5 to 50 mL under stirring conditions. N,N′-Dicyclohexylcarbodiimide (DCC) was added as a catalyst, and the system was refluxed for approximately 24 h. Thereafter, the system was allowed to cool to room temperature with continuous stirring. After cooling, amine modified graphene oxide 200 was separated by a centrifuge.

The amine modified graphene has a chemical structure as follows.

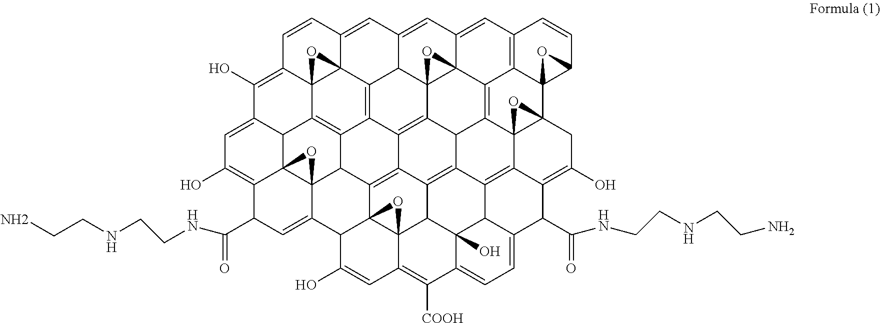

### Example 3—Synthesis of Tetradecanoic and Amine Modified Graphene Nanosheets Inhibitor

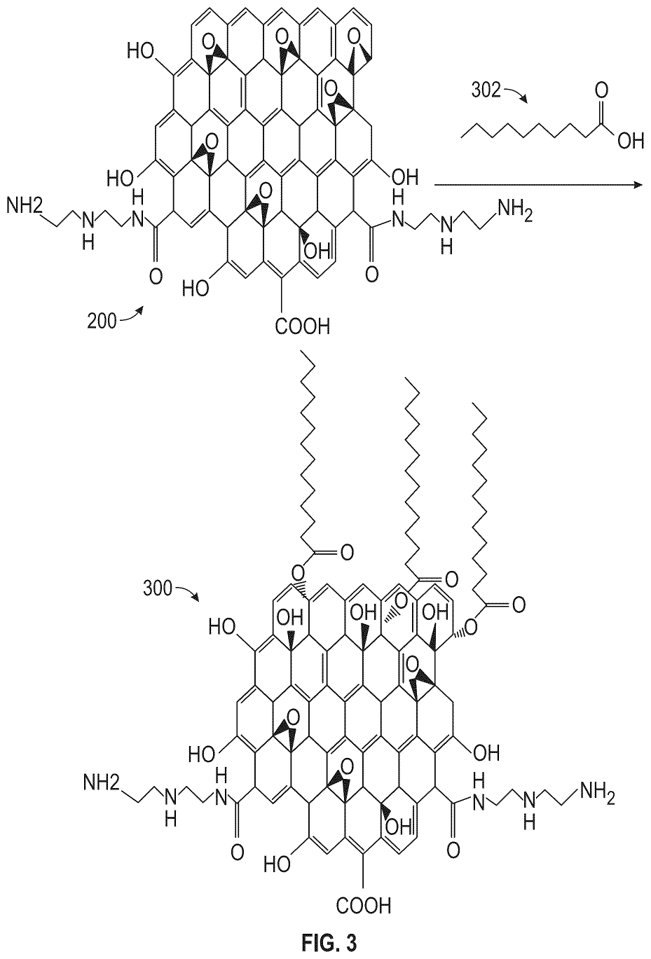

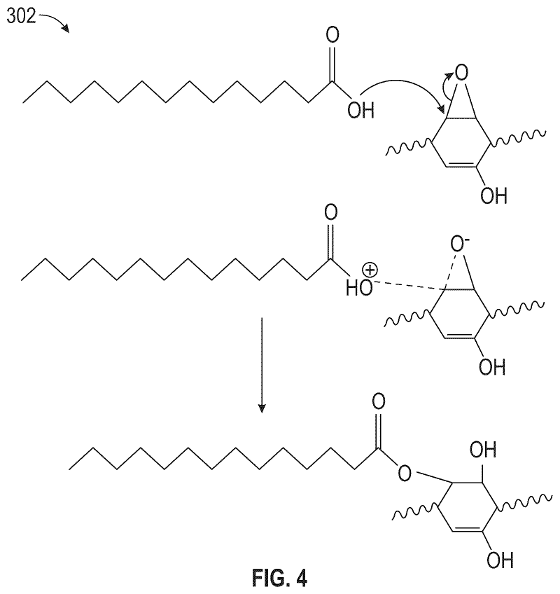

FIG. 3 shows the synthesis of tetradecanoic and amine modified graphene nanosheets 300. The obtained amine modified graphene oxide 200 was added into tetradecanoic acid 302 under stirring conditions in a graphene to tetradecanoic acid ratio ranging from 1:2 to 1:10. Then, small amounts of cobalt-salen catalyst, such as 0.1 g, was added, and the system was refluxed at a temperature of about 120° C. for approximately 6 h to allow the reaction to be completed. The reaction mechanism of the formation of tetradecanoic branches via epoxy groups and proton exchange on graphene is illustrated in FIG. 4. The product was collected and dried using a freeze drier to produce the tetradecanoic and amine modified graphene nanosheets 300.

The chemical structure of the tetradecanoic and amine modified graphene nanosheets is as follows.

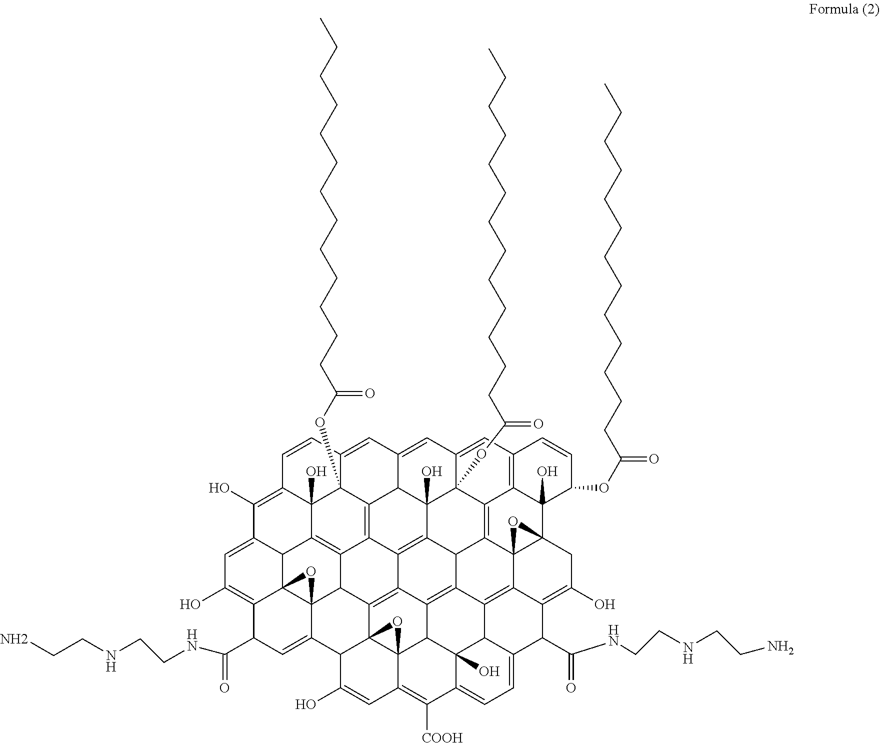

### Example 4—Weight Loss Measurement Experiment

The ASTM G1-03 methodology, which is the standard practice for preparing, cleaning, and evaluating corrosion test specimens, was used for a weight loss measurement trial. Pre-weighed carbon steel specimens were immersed entirely in duplicates in 100 ml of the test solutions housed in a 250 ml glass container held at ambient temperature (25±1° C.) for 24 hours. Each test sample was then removed, carefully cleansed, rinsed with distilled water and acetone, dried, and weighed. The difference in weight before and after the specimens were immersed in the test solutions was utilized to calculate the weight loss. The average weight loss was used to compute the corrosion rate.

Table 1 shows the weight loss measurement results for a 5% HCl blank compared with modified graphene inhibitor test solutions at a concentration of 300 parts per million (ppm) of modified graphene at 90° C. Table 2 shows the weight loss measurement results for a blank mixture acid system of 5% MSA+5% HCl compared with modified graphene inhibitor test solutions at a concentration of 300 ppm of modified graphene at 90° C.

TABLE 1

| | | Wt. Before | Wt. After | Weight | |
|---|---|---|---|---|---|
| Item | Acid System | (g) | (g) | Loss (g) | % IE |
| Blank | 5% HCl | 12.0376 | 10.189 | 1.8486 | — |
| 300 ppm | Inhibited | 11.9916 | 11.720 | 0.2716 | 97.74% |
| modified | 5% HCl | | | | |
| graphene | | | | | |

TABLE 2

| | | Wt. Before | Wt. After | Weight | |
|---|---|---|---|---|---|
| Item | Acid System | (g) | (g) | Loss (g) | % IE |
| Blank | 5% MSA + 5% | 12.0119 | 9.991 | 2.0209 | — |
| | HCl | | | | |
| 300 ppm | Inhibited 5% | 12.017 | 11.671 | 0.346 | 97.12% |
| modified | MSA + 5% | | | | |
| graphene | HCl | | | | |

The percent inhibition efficiency (% IE) was calculated according to the following:

$$\% {IE}_{Wt Loss} = \frac{{CR}_{o} - {CR}_{I}}{{CR}_{o}} \times 100 , \qquad ( 1 )$$

where CRₒ and CR_{I} are the weight before and after for the blank and inhibited test solutions, respectively.

The corrosion resistance of the corrosion inhibitor described herein against carbon steel corrosion was evaluated using a weight loss method in two acid systems, 5% hydrochloride (HCl) and 5% methanesulfonic acid (MSA)+5% HCl. As shown in Tables 1 and 2, the corrosion rate value for the inhibited test solutions was significantly lower than the blank 5% HCl at 90° C. (Table 1). Moreover, the 300 ppm modified graphene inhibited solutions corroded at a slower rate than the blank mixture of acid system 5% MSA+5% HCl at 90° C., as indicated in Table 2. Additionally, there was a significant inhibition efficiency 97.74% and 97.12% for both the 5% HCl solution and the 5% MSA+5% HCl solution at 90° C., respectively. The results indicate that the corrosion inhibitor described herein significantly reduces the corrosion rate when HCl is used for acid stimulation at high temperatures. Additionally, the corrosion inhibitor according to embodiments of the present disclosure may mitigate iron sulfide deposition in formation and downhole tubing in carbonate sour gas and oil wells through effectively controlling acid corrosion during acidizing treatment.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures.

1. A method of synthesizing a corrosion inhibitor, comprising:
synthesizing amine modified graphene oxide from graphene oxide using a bifunctional amine comprising a terminal amide group.

2. The method of claim 1, comprising synthesizing aliphatic and amine modified graphene oxide from the amine modified graphene oxide using an aliphatic acid, wherein the aliphatic and amine modified graphene oxide comprises aliphatic groups derived from a long chain aliphatic acid and amine groups derived from a bifunctional amine.

3. The method of claim 1, wherein the graphene oxide comprises graphene oxide nanosheets.

4. The method of claim 2, wherein the aliphatic and amine modified graphene oxide comprises long chain aliphatic and amine modified graphene oxide nanosheets.

5. (canceled)

6. The method of claim 1, wherein the amine modified graphene oxide has a chemical structure shown in Formula (2).

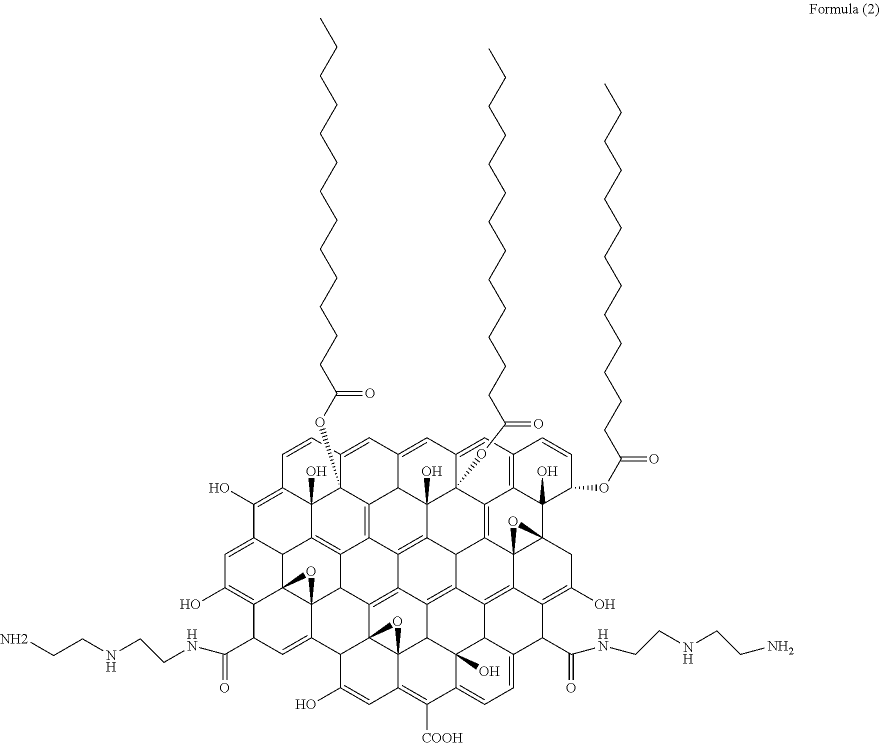

7. (canceled)

8. The method of claim 6, wherein preparing the graphene oxide comprises:
mixing an amount of graphite powder with an amount of potassium permanganate, forming a first solution;
mixing an amount of sulfuric acid with an amount of phosphoric acid, forming a second solution;
stirring the first solution into the second solution, forming a third solution;
heating and stirring the third solution for a predetermined amount of time; and
cooling the third solution and adding an amount of hydrogen peroxide to the cooled solution.

9.-16. (canceled)
